(19) United States
(12) Patent Application Publication
(10) Pub. No.: US 2018/0305650 A1
(43) Pub. Date: Oct. 25, 2018

(54) Culture Bag and Culture Device

(71) Applicant: DEXERIALS CORPORATION, Shinagawa-ku, Tokyo (JP)

(72) Inventors: Akira Higuchi, Kyoto-shi, Kyoto (JP); Hiroyuki Naito, Kyoto-shi, Kyoto (JP); Keiji Honjo, Shinagawa-ku, Tokyo (JP); Toru Abiko, Shinagawa-ku, Tokyo (JP); Kazuya Hayashibe, Shinagawa-ku, Tokyo (JP); Yasuyuki Kudo, Shinagawa-ku, Tokyo (JP); Hirokazu Odagiri, Shinagawa-ku, Tokyo (JP)

(21) Appl. No.: 15/779,346

(22) PCT Filed: Nov. 25, 2016
(86) PCT No.: PCT/JP2016/085061

§ 371 Date: May 25, 2018

(30) Foreign Application Priority Data
Nov. 27, 2015 (JP) 2015-232251

(51) Int. Cl. C12M 1/00; B01F 11/00; B01F 15/00; C12M 3/00; C12M 1/02

(52) U.S. Cl. CPC C12M 23/14; B01F 11/0025; B01F 15/0085; B01F 2215/0073; C12M 23/50; C12M 27/00; C12M 23/48

(57) ABSTRACT

A culture bag includes a culture portion which has a culture space configured to contain and culture a culture fluid. The culture space is an endless circumferential circulation space in which the culture fluid can circumferentially circulate. The culture fluid can continue to circumferentially circulate in one direction in the endless culture space. This suppresses complexification of flow of the culture fluid. As a result, bubbles can be suppressed from generating without decreasing culture efficiency.

### TECHNICAL FIELD

The present invention relates to a culture bag and a culture device which are used for culturing, for example, microorganisms or animal or plant cells.

### BACKGROUND ART

Hitherto, disposable culture bags have been used for culturing, for example, microorganisms or animal or plant cells. The culture bags are in a bag form containing a culture fluid in which a culture target (e.g., cells) is suspended at a certain concentration (number), for example, as described in PTL 1. The culture bag is provided with a port configured to supply a mixed gas, for example, oxygen and carbon dioxide having a controlled concentration into the culture bag, a port configured to supply or recover the culture fluid, and a port configured to take a sample. Such culture bags are formed of an elastomeric material and are kept in a defined shape in use by the action of pressure of the mixed gas.

Moreover, such culture bags are periodically changed in its position and posture in order to facilitate culturing, for example, facilitate cell proliferation. For example, the culture bag described in PTL 1 is fixed onto a stage which swings about a swinging axis. An amount of the mixed gas to be introduced into the culture fluid, for example, a dissolved oxygen amount is determined depending on swinging conditions of the culture bag, that is, a swinging stroke, a swinging angle, and a swinging rate. The swinging conditions are determined depending on properties of the culture target (e.g., cells).

Thus, the culture fluid flows to thereby create waves on its liquid surface, so that the liquid surface (gas-liquid interface) is increased in area. The thus-created waves of a culture wall are broken by colliding an inner wall surface of the culture bag. As a result, the mixed gas is actively introduced into the culture fluid. Moreover, the culture fluid is stirred and the thus-introduced gas spreads throughout the culture fluid, leading to facilitation of cell proliferation in the culture fluid.

### CITATION LIST

### Patent Literature

PTL 1 Japanese Patent Application Laid-Open (JP-A) No. 2010-540228

### SUMMARY OF INVENTION

### Technical Problem

Depending on types of the culture target and the swinging conditions of the culture bag, a gas involved in collision between the waves of the culture fluid and the inner wall surface of the culture bag may turn into bubbles. In particular, when big waves of the culture fluid are created, the bubbles are generated.

When the bubbles are broken, impact resulting therefrom damages cells around the bubbles, potentially leading to cell death. Moreover, the bubbles may aggregate into large bubbles (foam) to thereby inhibit the mixed gas from dissolving in the culture fluid.

When the waves of the culture fluid collide the inner wall surface of the culture bag to thereby rapidly change a flow direction of the culture fluid, shear stress is caused. When the thus-caused shear stress is high, cells may be damaged. The bigger the waves of the culture fluid are, the higher the shear stress is, that is, the greater extent the cells are damaged.

Therefore, the bigger the waves of the culture fluid are, the greater extent culturing of the culture target (e.g., proliferation of cells) may be inhibited.

In order to suppress such waves of the culture fluid, which may inhibit the culturing, from being created, it is contemplated that a position and a posture of the culture bag is suppressed from periodically changed, that is, an amount of the change is decreased and a period of the change is prolonged. For example, in the case of the culture device described in PTL 1, it is contemplated that a swinging stroke amount of a stage on which the culture bag is placed is decreased and a swinging rate thereof is slowed down.

However, in this case, although the waves of the culture fluid, which may inhibit such a culturing, can be suppressed from being created, an amount of a gas to be introduced into the culture fluid such as oxygen may consequently be insufficient, potentially leading to lower culturing efficiency of the culture target (e.g., a lower proliferation rate of cells).

Note that, in addition to the culturing using the culture bag, there have been culturing methods using Erlenmeyer flasks. In the case of the Erlenmeyer flask, the Erlenmeyer flask is periodically changed in position (is allowed to revolve) so that a culture fluid therein circumferentially circulates. Therefore, there is substantially no collision between an inner wall surface of the Erlenmeyer flask and waves of the culture fluid. In addition, the thus-created waves of the culture fluid are small.

However, in the case of the Erlenmeyer flask, because it is limited in size, a culturing volume (culturing scale) is limited to several liters. Moreover, because there is a large difference in flow velocity of the culture fluid between the proximity of the inner wall surface and the proximity of the center, the culture target (e.g., cells) aggregates in the proximity of the center of the Erlenmeyer flask at which the flow velocity is almost zero to thereby damage the culture target. Therefore, in the case of the Erlenmeyer flask, although the waves of the culture fluid, which may inhibit the culturing, can be suppressed from being created, the culturing efficiency is lower (e.g., compared to that of a culture bag which allows culturing in a scale of about 50 liters).

Therefore, an object of the present invention is to suppress waves of a culture fluid, the waves creating bubbles and shear stress which may damage a culture target, from being created without decreasing culture efficiency in a culturing which is performed while the culture fluid is flowing in a culture bag.

### Solution to Problem

In order to solve the above-described technical problems, according to one aspect of the present invention, provided is a culture bag including a culture portion which has a culture space configured to contain and culture a culture fluid; wherein the culture space is an endless circumferential circulation space in which the culture fluid can circumferentially circulate.

According to another aspect of the present invention, provided is a culture device including a culture bag which includes a culture portion having a culture space configured to contain and culture a culture fluid, the culture space being an endless circumferential circulation space in which the culture fluid can circumferentially circulate; a stage configured to hold the culture bag; and a stage driving portion configured to change a position and a posture of the stage so that the culture fluid circumferentially circulates in the culture space of the culture bag.

According to additional another aspect of the present invention, provided is a culture device including a culture bag having a culture space configured to contain and culture a culture fluid; a stage configured to hold the culture bag; a culture space deforming portion configured to deform the culture space into an annular space; and a stage driving portion configured to change a position and a posture of the stage so that the culture fluid circumferentially circulates in the thus-deformed annular culture space of the culture bag.

### Advantageous Effects of the Invention

According to the present invention, waves of a culture fluid, the waves creating bubbles and shear stress which may damage a culture target, can be suppressed from being created without decreasing culture efficiency in a culturing which is performed while the culture fluid is flowing in a culture bag.

### BRIEF DESCRIPTION OF DRAWINGS

FIG. 1 is a schematic perspective view of a culture device according to one embodiment of the present invention.

FIG. 2 is a schematic perspective view of a culture bag according to one embodiment of the present invention.

FIG. 3 is a top view of a culture bag.

FIG. 4 is a longitudinal sectional view taken along the Yb axis in FIG. 3.

FIG. 5 is a longitudinal sectional view taken along the Xb axis in FIG. 3.

FIG. 6 is a top view of a tray on which a culture bag is held.

FIG. 7 is a longitudinal sectional view taken along the Yb axis in FIG. 6.

FIG. 8 is a block diagram illustrating a control system of a culture device.

FIG. 9 is a time-chart illustrating one exemplary control for circumferentially circulating a culture fluid in an annular culture space.

FIG. 10 is a time-chart illustrating another exemplary control for circumferentially circulating a culture fluid in an annular culture space.

FIG. 11 is a time-chart illustrating additional another exemplary control for circumferentially circulating a culture fluid in an annular culture space.

FIG. 12 is a time-chart illustrating a different exemplary control for circumferentially circulating a culture fluid in an annular culture space.

FIG. 13 is a time-chart illustrating a different control for circumferentially circulating a culture fluid in an annular culture space, the control changing over time.

FIG. 14 is a top view of a culture bag according to another embodiment.

FIG. 15 is a top view of a culture bag according to additional another embodiment.

FIG. 16 is a top view of a culture bag according to a different embodiment.

FIG. 17 is a longitudinal sectional view of a culture bag according to an additional different embodiment.

FIG. 18 is a longitudinal sectional view of a culture bag according to an additional further different embodiment.

FIG. 19 is a top view schematically illustrating a configuration of a culture device according to another embodiment.

FIG. 20 is a sectional view taken along the Xb axis illustrated in FIG. 19.

FIG. 21 is a sectional view for explaining a configuration of a modified form of a culture device illustrated in FIG. 19.

### DESCRIPTION OF EMBODIMENTS

A culture bag according to one aspect of the present invention includes a culture portion which has a culture space configured to contain and culture a culture fluid. The culture space is an endless circumferential circulation space in which the culture fluid can circumferentially circulate.

According to this aspect, the culture fluid can circumferentially circulate in the endless culture space. Circumferential circulation of the culture fluid suppresses collision between an inner wall surface of the culture space and the culture fluid and creates smaller waves. Moreover, because the culture space is an endless circumferential circulation space in which the culture fluid can circumferentially circulate, a region of which flow velocity is almost zero (so-called stagnancy) is suppressed from occurring in the culture fluid. Therefore, a culture target is suppressed from aggregating in the region of which flow velocity is almost zero. As a result, waves of the culture fluid can be suppressed from being created without decreasing culture efficiency, the waves creating bubbles and shear stress which may damage the culture target.

The culture space in the culture bag may be annular.

A longitudinal section perpendicular to a circumferential circulation direction of the culture space in the culture bag may be circular. This allows the culture fluid to smoothly flow in a circumferential direction of the longitudinal section along the inner wall surface of the culture space, making it more difficult to create shear stress which results from a rapid change of a flow direction.

The culture portion of the culture bag may have a double bag structure including an inner bag portion and an outer bag portion configured to contain the inner bag portion. An inner space of the inner bag portion may be the culture space. A space between the inner bag portion and the outer bag portion may be a gas-containing space configured to contain a gas. The inner bag portion may be configured to contain the culture fluid in the inner space and be gas-permeable. This allows the gas to be supplied in a form of microbubbles into the culture fluid within the culture space to thereby decrease the necessity to make the culture fluid flow (the culture fluid is enough only to flow to an extent necessary for facilitating the culturing). As a result, it is more difficult to create waves of the culture fluid CF, the waves creating bubbles and shear stress which may damage the culture target.

A culture device according to another aspect of the present invention includes a culture bag which includes a culture portion having a culture space configured to contain and culture a culture fluid, the culture space being an endless circumferential circulation space in which the culture fluid can circumferentially circulate; a stage configured to hold the culture bag; and a stage driving portion configured to change a position and a posture of the stage so that the culture fluid circumferentially circulates in the culture space of the culture bag.

According to this aspect, the culture fluid can circumferentially circulate within the endless culture space. As a result, waves of the culture fluid, the waves creating bubbles and shear stress which may damage the culture target, can be suppressed from being created without decreasing the culture efficiency.

A culture device according to additional another aspect of the present invention includes a culture bag having a culture space configured to contain and culture a culture fluid; a stage configured to hold the culture bag; a culture space deforming portion configured to deform the culture space into an annular space; and a stage driving portion configured to change a position and a posture of the stage so that the culture fluid circumferentially circulates in the thus-deformed annular culture space of the culture bag.

According to this aspect, the culture fluid can circumferentially circulate within the endless culture space. As a result, waves of the culture fluid, the waves creating bubbles and shear stress which may damage the culture target, can be suppressed from being created without decreasing the culture efficiency.

Embodiments of the present invention will now be described with reference to drawings.

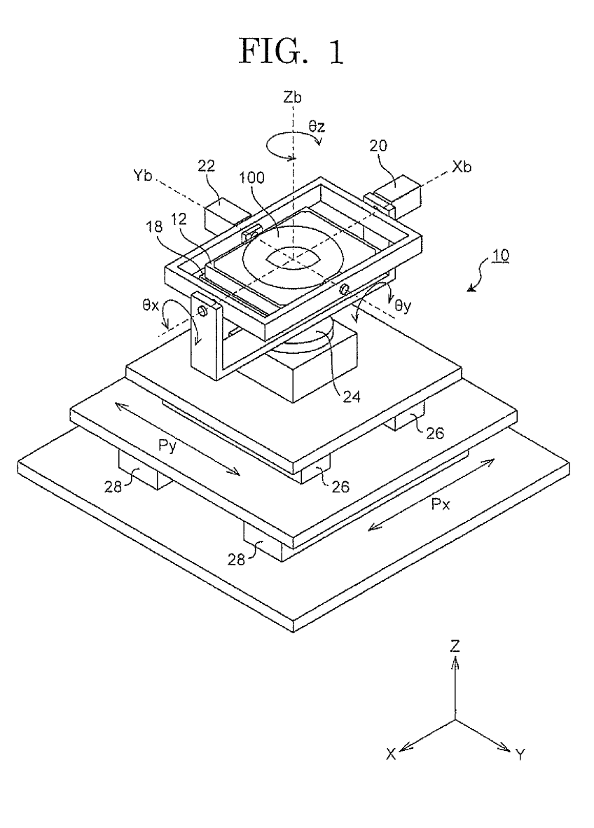

FIG. 1 schematically illustrates a culture device according to one embodiment of the present invention. Note that, in this drawing, the X-Y-Z rectangular coordinate system is illustrated, which is intended to facilitate understanding of embodiments of the present invention but is not intended to limit the present invention. The X-axis direction and the Y-axis direction are horizontal directions and the Z-axis direction is a vertical direction.

Note that, briefly, the culture device according to embodiments of the present invention includes a culture bag having an (e.g., doughnut-like) endless circumferential circulation space (culture space) in which a culture fluid can circumferentially circulate and is configured to change a position and a posture of the culture bag so that the culture fluid circumferentially circulates in the culture space of the culture bag. Details will now be described.

A culture device 10 illustrated in FIG. 1 is a device configured to change a position and a posture of a culture bag 100 in order to facilitate culturing within the culture bag 100. First, the culture bag 100 will now be described.

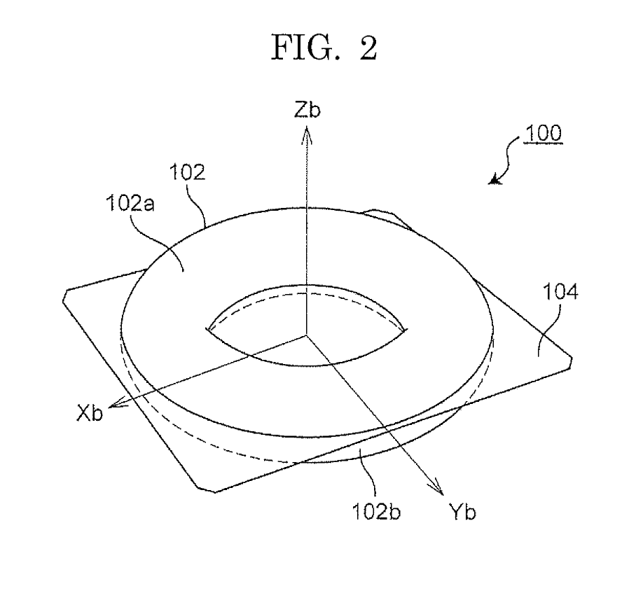

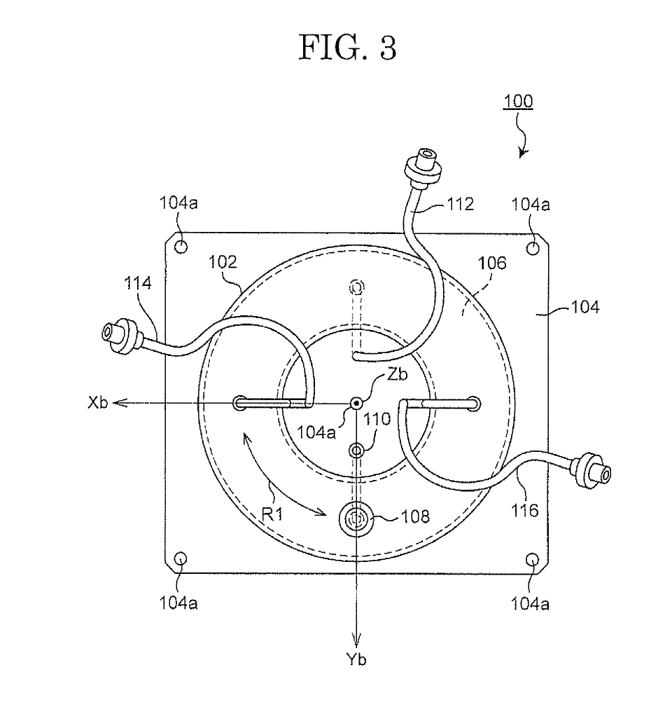

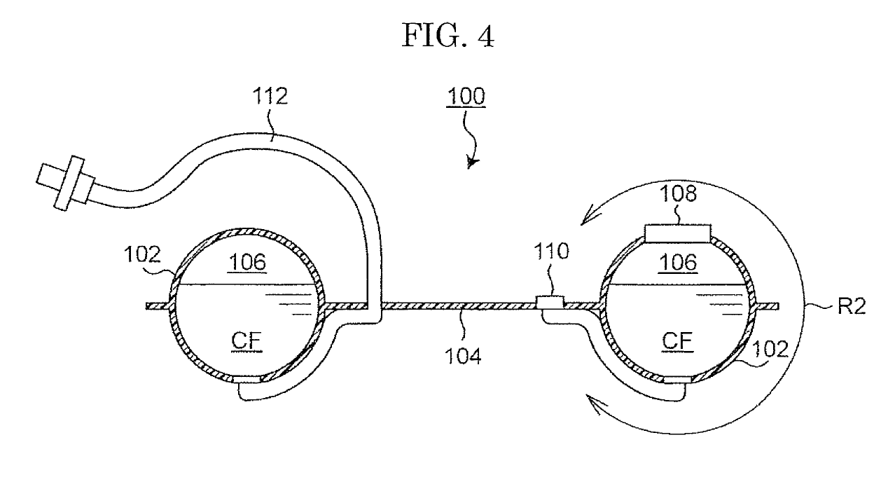

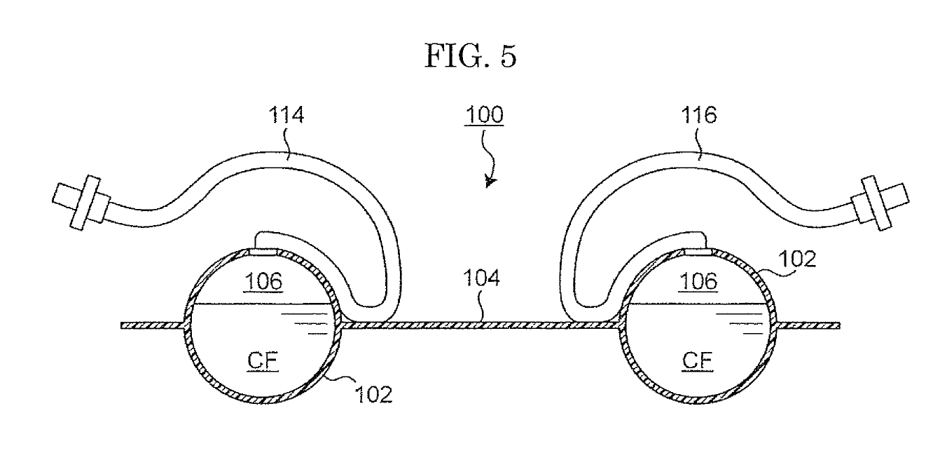

FIG. 2 is a schematic perspective view of the culture bag 100. FIG. 3 is a top view of the culture bag 100. FIG. 4 is a cross-sectional view taken along the Yb axis in FIG. 3. FIG. 5 is a cross-sectional view taken along the Xb axis in FIG. 3.

As illustrated in FIG. 2, the culture bag 100 is in a bag form in which microorganisms or cells are cultured with the culture fluid. In the case of the present embodiment, the culture bag 100 is made of a flexible material such as polyethylene and elastomeric materials so as to be compressed upon disposal taking into consideration of being single-use.

The culture bag 100 includes a culture portion 102 configured to contain a culture fluid in which a culture target (e.g., cells) is suspended at a certain concentration (number) to thereby culture microorganisms or cells and a sheet-like bracket portion 104 configured to hold the culture portion 102.

As illustrated in FIG. 3, the culture portion 102 of the culture bag 100 has a culture space 106 configured to contain and culture the culture fluid. In the case of the present embodiment, the culture space 106 is an endless circumferential circulation space in which the culture fluid can circumferentially circulate, and is an annular, in particular, a circularly annular (doughnut-like) space including a circular longitudinal section.

Note that, some terms with respect to the annular culture space 106 will now be defined. First, a circumferential circulation direction of the annular culture space 106, which is a circumferential circulation space, is defined as R1. An axis perpendicular to a plane including the circumferential circulation direction R1 is defined as a third bag axis Zb. The axes included in the plane including the circumferential circulation direction R1 and perpendicular to the third bag axis Zb and to each other are defined as first and second bag axes Xb and Yb. A circumferential direction of the longitudinal section in the culture space 106, the circumferential direction being perpendicular to the circumferential circulation direction R1, is defined as a longitudinal section circumferential direction R2.

Moreover, in the case of the present embodiment, because the culture space 106 is circularly annular, the third bag axis Zb is a central axis passing through the center of the circularly annular culture space. The sheet-like bracket portion 104 is expanded along the first and second bag axes Xb, Yb.

The bracket portion 104 configured to hold the culture portion 102 of the culture bag 100 serves as a bracket configured to attach the culture bag 100 to the culture device 10. Therefore, in the case of the present embodiment, the bracket portion 104 of the culture bag 100 is provided with a plurality of through holes 104a which are used for screwing the bracket portion to the culture device 10.

Note that, in the case of the present embodiment, as illustrated in FIG. 2, the culture portion 102 is disposed in the bracket portion 104 so as to penetrate the bracket portion 104. That is, the culture portion 102 is divided by the bracket portion 104 into an upper half 102a (portion located on the upper side in the state when attached to the culture device 10) and a lower half 102b. However, the culture space 106 of the culture portion 102 penetrates the bracket portion 104.

Moreover, in the case of the present embodiment, a plurality of ports (horses) 108, 110, 112, 114, and 116 are disposed in the culture portion 102 of the culture bag 100.

Each of the plurality of ports 108, 110, 112, 114, and 116 is in communication with the culture space 106 of the culture portion 102.

The culture fluid port 108 is a port used for supplying the culture fluid CF to the culture space 106 of the culture portion 102 and for recovering the culture fluid CF from the culture space 106. The culture fluid port 108 is disposed in the upper half 102a of the culture portion 102.

A sampling port 110 is used for taking samples of microorganisms or cells cultured in the culture space 106 of the culture portion 102. An indicated amount of the culture fluid (e.g., cell suspension) can be taken from the culture bag 100 via the port 110. The thus-taken suspension can be observed by, for example, a microscope to thereby verify the degree of progress in culturing. For example, the degree of cell growth can be determined by counting the number of cells by means of a microscope. Note that, the sampling port 110 is a port including, for example, a valved lues lock connector. The sampling port 110 extends from the lower half 102b of the culture portion 102 and is opened at the bracket portion 104.

The first gas supplying port 112 is a port used for supplying a mixed gas necessary for culturing such as oxygen and carbon dioxide into the culture space 106 of the culture portion 102. The gas supplying port 112 extends from the lower half 102b of the culture portion 102.

The exhaust port 114 is a port used for exhausting the culture space 106 of the culture portion 102 or controlling pressure in the culture space 106 by exhausting. The exhaust port 114 extends from the upper half 102a of the culture portion 102.

The second gas supplying port 116 is a port used, like the first gas supplying port 112, for supplying a mixed gas necessary for culturing such as oxygen and carbon dioxide into the culture space 106 of the culture portion 102. The second gas supplying port 116 extends from the upper half 102a of the culture portion 102. As described below in detail, in the case of the present embodiment, the second gas supplying port 116 is mainly used and the first gas supplying port 112 is auxiliary used.

Note that, positions of the circumferential circulation direction R1 and the longitudinal section circumferential direction R2 on the culture portion 102 on which the plurality of ports 108, 110, 112, 114, and 116 are disposed may be changed depending on applications of the culture bag 100 (types of culturing). Moreover, a filter is disposed in the first and second gas supplying ports 112, 116 and the exhaust port 114 in order to suppress contaminants from entering into the culture space 106 of the culture bag 100.

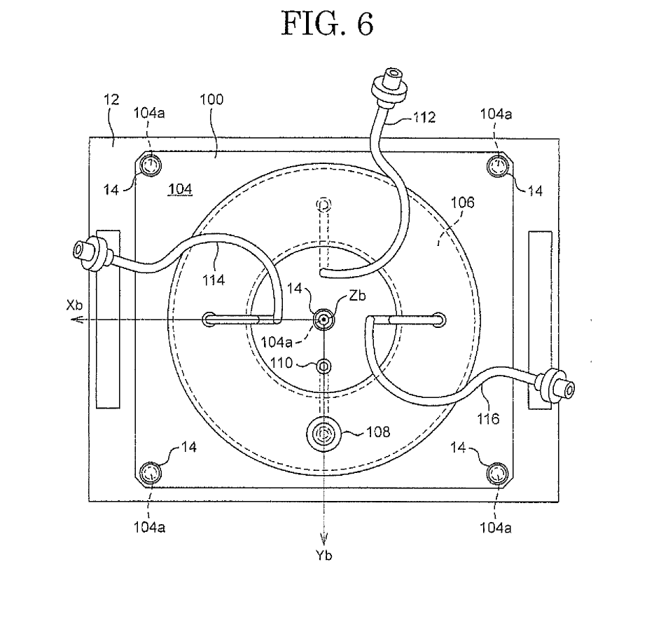

In the case of the present embodiment, as illustrated in FIG. 6, the culture bag 100 is attached to the culture device 10 with being fixed to the tray 12. The culture bag 100 is fixed to the tray 12 via a plurality of knurled screws 14 penetrating the plurality of through holes 104a formed on the bracket portion 104.

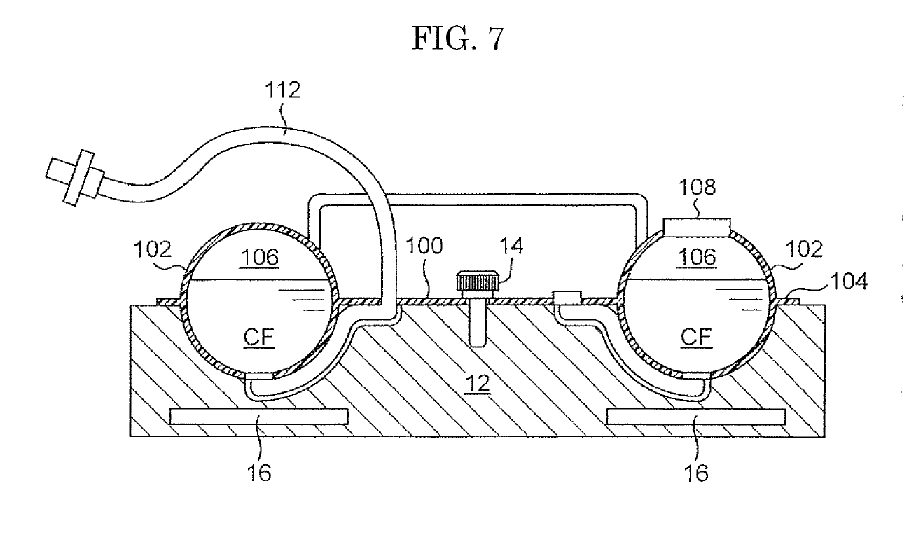

As illustrated in FIG. 7 illustrating the cross section taken along the Yb axis illustrated in FIG. 6, a heater 16, which is configured to control a temperature in the culture space 106 of the culture portion 102 in the culture bag 100, is disposed in the tray 12.

As illustrated in FIG. 1, the culture device 10 includes a stage 18 on which the tray 12 is configured to be placed with being fixed thereto.

The culture device 10 includes a plurality of motors 20, 22, 24 and a plurality of actuators 26, 28 in order to change a position and a posture of the stage 18 (drive the stage 18), that is, to change a position and a posture of the culture bag 100 on the tray 12 placed on the stage 18.

The motor 20 is a motor configured to swing the culture bag 100 fixed to the stage 18 via the tray 12 about the first bag axis Xb of the culture bag 100.

The motor 22 is a motor configured to swing the culture bag 100 fixed to the stage 18 via the tray 12 about the second bag axis Yb of the culture bag 100.

The motor 24 is a motor configured to swing the culture bag 100 fixed to the stage 18 via the tray 12 about the third bag axis Zb of the culture bag 100.

Note that, the stage 18 is placed on the culture device 10 so that the culture bag 100 fixed to the stage 18 via the tray 12 can be shaken about the first, second, and third bag axes Xb, Yb, Zb.

The actuator 26 is an actuator configured to parallelly move the culture bag 100 fixed to the stage 18 via the tray 12 in the X axis direction (horizontal direction).

The actuator 28 is an actuator configured to parallelly move the culture bag 100 fixed to the stage 18 via the tray 12 in the Y axis direction (horizontal direction).

The position and the posture of the culture bag 100 fixed to the stage 18 via the tray 12 is changed by the motors 20, 22, 24 and the actuator 26, 28. This allows the culture fluid CF within the culture space 106 of the culture portion 102 of the culture bag 100 to flow in the culture space 106. In the case of the present embodiment, the position and the posture of the culture bag 100 is changed so that the culture fluid CF circumferentially circulates within the circularly annular culture space 106 in the circumferential circulation direction R1.

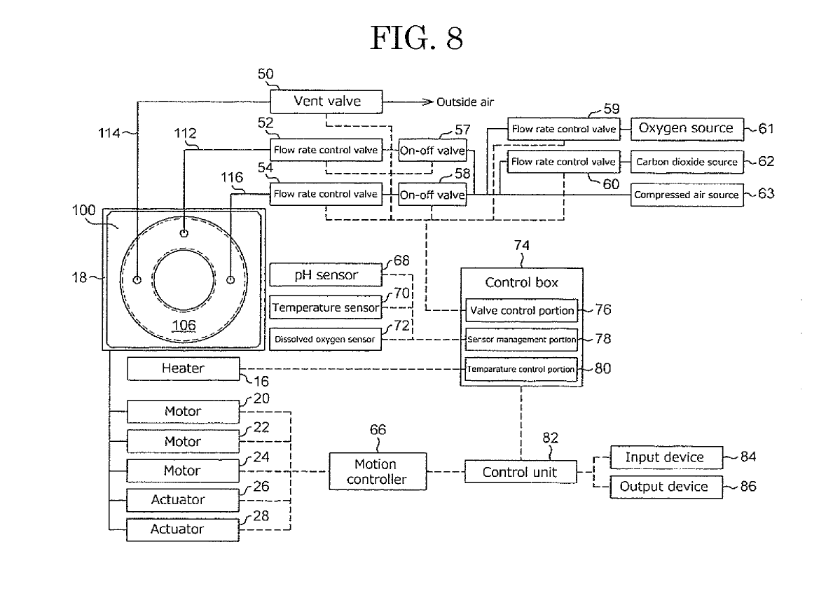

FIG. 8 is a block diagram illustrating a control system of the culture device 10 configured to perform culturing using the culture fluid CF in the state in which the culture fluid CF circumferentially circulates within the circularly annular culture space 106 of the culture bag 100 in the circumferential circulation direction R1.

As illustrated in FIG. 8, the culture device 10 includes a vent valve 50 configured to be connected to the exhaust port 114 of the culture bag 100, a flow rate control valve 52 configured to be connected to the first gas supplying port 112, and a flow rate control valve 54 configured to be connected to the second gas supplying port 116.

The vent valve 50 is a valve configured to control a pressure within the culture space 106 by discharging a gas from the culture space 106 of the culture bag 100 to the outside. For this purpose, the vent valve 50 is disposed between the exhaust port 114 of the culture bag 100 and the outside air. The pressure of the culture space 106 is controlled by controlling the degree of opening of the vent valve 50.

The flow rate control valves 52, 54 are valves configured to control an amount of a mixed gas of oxygen and carbon dioxide to be supplied into the culture space 106 of the culture bag 100. The flow rate control valve 52 is connected to the first gas supplying port 112 of the culture bag 100 and the flow rate control valve 54 is connected to the second gas supplying port 116.

The flow rate control valves 52, 54 are connected to an oxygen source (e.g., oxygen bomb) 61 and a carbon dioxide source (e.g., carbon dioxide bomb) 62 via on-off valves 57, 58 and flow rate control valves 59, 60.

Specifically, the flow rate control valve 52 is connected to a compressed air source (e.g., air bomb) 63 via the on-off valve 57. The flow rate control valve 54 is connected to the compressed air source 63 via the on-off valve 58. Moreover, the oxygen source 61 is connected to between the on-off valves 57, 58 and the compressed air source 63 via the flow rate control valve 59. The carbon dioxide source 62 is connected to between the on-off valves 57, 58 and the compressed air source 63 via the flow rate control valve 60.

Oxygen from the oxygen source 61 and carbon dioxide from the carbon dioxide source 62 are carried by the compressed air from the compressed air source 63 and mixed with each other. The mixed gas carried by the compressed air is sent to only the flow rate control valve 54 or both of the flow rate control valves 52, 54, that is, is sent to only the second gas supplying port 116 or both of the first and second gas supplying ports 112, 116. Amounts of the oxygen and the carbon dioxide in the mixed gas are controlled by changing the degree of opening of the flow rate control valves 59, 60. By selective opening or closing the on-off valves 57, 58, the mixed gas is supplied into only the second gas supplying port 116 via only the flow rate control valve 54 or supplied into both of the first and second gas supplying ports 112, 116 via both of the flow rate control valves 54, 56. Moreover, by changing the degree of opening of each of the flow rate control valves 52, 54, an amount of the mixed gas to be supplied into the first and second gas supplying ports 112, 116 is controlled.

Thus, the oxygen and the carbon dioxide are supplied into the culture space 106 of the culture bag 100 via the second gas supplying port 116, the amount of the oxygen to be supplied into the culture space 106 by the flow rate control valves 54, 59, and 60, that is, an oxygen concentration within the culture fluid CF is controlled, and the amount of the carbon dioxide to be supplied into the culture space, that is, a pH value of the culture fluid CF is controlled. Moreover, the culture portion 102 of the culture bag 100 (culture space 106) is kept in an approximately constant shape by the action of the compressed air.

When the oxygen concentration and the pH value of the culture fluid CF are lower than the set value, the on-off valve 57 is opened to additionally supply the mixed gas of oxygen and carbon dioxide into the culture space 106 of the culture bag 100 via the first gas supplying port 112. Thus, the oxygen concentration and the pH value of the culture fluid CF can be carefully controlled by including the plurality of ports configured to supply the mixed gas into the culture space 106 of the culture bag 100 (in the case of the present embodiment, the first and second gas supplying ports 112, 116).

Note that, when the culture space 106 of the culture bag 100 is filled with the culture fluid CF, the second gas supplying port 116 and the exhaust port 114 are not used.

The culture device 10 includes a motion controller 66 configured to control the motors 20, 22, 24 and the actuators 26, 28 to change a position and a posture of the stage 18, that is, to control behavior of the culture bag 100. The motion controller 66 is, for example, a circuit board configured to supply electric power for driving the motors 20, 22, 24 and the actuators 26, 28 to the motors and the actuators so that the culture fluid CF circumferentially circulates within the circularly annular culture space 106 of the culture bag 100.

The culture device 10 includes a pH sensor 68, a temperature sensor 70, and a dissolved oxygen sensor 72 in order to monitor a state of the culture fluid CF in culture. The pH sensor 68 is configured to detect a pH valve of a solvent fluid CF within the culture space 106, the temperature sensor 70 is configured to detect a temperature of the solvent fluid CF, and the dissolved oxygen sensor 72 is configured to detect an oxygen concentration in the solvent fluid CF.

Based on the state of the culture fluid CF in culture, that is, detection results of the pH sensor 68, the temperature sensor 70, and the dissolved oxygen sensor 72, the culture device 10 includes a control box 74 configured to control the vent valve 50, the flow rate control valves 52, 54, 59, and 60, the on-off valve 57 and 58, and the heater 16. The control box 74 includes a valve control portion 76 configured to control the plurality of valves 50, 52, 54, and 57 to 60, a sensor management portion 78 configured to acquire detection values of the pH sensor 68, the temperature sensor 70, and the dissolved oxygen sensor 72, and a temperature control portion 80 configured to control the heater 16.

First, the sensor control portion 78 of the control box 74 is connected to each of the pH sensor 68, the temperature sensor 70, and the dissolved oxygen sensor 72 and is configured to periodically acquire the pH value of the culture fluid CF detected by the pH sensor 68, the temperature of the culture fluid CF detected by the temperature sensor 70, and the oxygen concentration of the solvent fluid CF detected by the dissolved oxygen sensor 72.

The valve control portion 76 is configured to control the plurality of valves 50, 52, 54, and 57 to 60 so as to keep each of the pH value and the oxygen concentration of the solvent fluid CF acquired by the sensor management portion 78 at the set value. The temperature control portion 80 is configured to control the heater 16 so as to keep the temperature of the solvent fluid CF acquired by the sensor management portion 78 at the set value.

Culturing environment (the pH value, the temperature, and the oxygen concentration of the culture fluid CF) set by users is kept by the action of the valve control portion 76, the sensor control portion 78, and the temperature control portion 80. Note that, the valve control portion 76, the sensor control portion 78, and the temperature control portion 80 are, for example, circuit boards configured to be capable of outputting control signals (electric current) to each of the plurality of valves 50, 52, 54, and 57 to 60, to be capable of receiving detection signals (electric current) from the pH sensor 68, the temperature sensor 70, and the dissolved oxygen sensor 72, and to be capable of supplying driving electric power to the heater 16.

The culture device 10 includes a control unit 82 for allowing users to set culturing conditions. The control unit 82 is, for example, a computer and includes an input device 84 configured to input the culturing conditions desired by the users such as a mouse and a keyboard and an output device 86 configured to allow the users to confirm the culturing conditions and a state in culture such as a display. The control unit 82 is configured to allow the motion controller 66 to change the position and the posture (behavior) of the culture bag 100 as set by the users via the input device 84 and to instruct the control box 74 to keep the culturing conditions (the pH value, the temperature, and the oxygen concentration of the culture fluid CF) as set by the users via the input device 84.

An example of controlling the motors 20, 22, 24 and the actuators 26, 28 for circumferentially circulating the culture fluid CF within the circularly annular culture space 106 of the culture bag 100 in the circumferential circulation direction R1 will now be described.

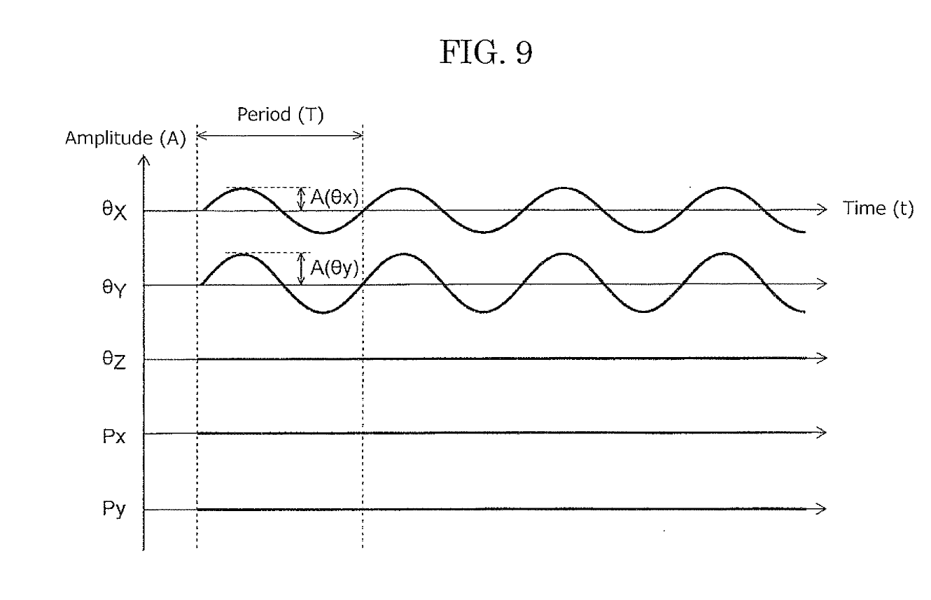

FIG. 9 is a time-chart illustrating one exemplary control for circumferentially circulating the culture fluid CF within the circularly annular culture space 106 of the culture bag 100.

In this drawing, as illustrated in FIG. 1, θx denotes a rotation angle of the culture bag 100 about the first bag axis Xb of the culture bag 100, the rotation angle being created by the motor 22; θy denotes a rotation angle of the culture bag 100 about the second bag axis Yb, the rotation angle being created by the motor 20; and θz denotes a rotation angle of the culture bag 100 about the third bag axis Zb, the rotation angle being created by the motor 24. Note that, when θx=θy=θz=0, the first bag axis Xb extends horizontally (in parallel to the X axis), the second bag axis Yb extends horizontally (in parallel to the Y axis), and the third bag axis Zb extends vertically (in parallel to the Z axis).

In this drawing, Px denotes a position of the culture bag 100 in the X axis direction and Py denotes a position of the culture bag 100 in the Y axis direction.

Note that, when θx=θy=θz=Px=Py=0, the stage 18, that is, the culture bag 100 on the stage 18 is present at an initial position.

In the example illustrated in FIG. 9, only the motor 20, which is configured to swing the culture bag 100 about the first bag axis Xb, and the motor 22, which is configured to swing the culture bag 100 about the second bag axis Yb, are used for circumferentially circulating the culture fluid CF within the circularly annular culture space 106 of the culture bag 100. That is, the rotation angles θx, θy periodically change and the rotation angle θz, the position in the X axis direction Px, and the position in the Y axis direction Py are kept zero (the origin).

Because the rotation angles θx, θy change in the same period and the same phase but in different amplitudes A (θx), A (θy), the culture fluid CF circumferentially circulates within the circularly annular culture space 106 in one circumferential circulation direction R1 at the approximately constant rate. Note that, when a turbulent flow is intendedly generated in order to facilitate culturing, periods T thereof may be different from each other. The amplitudes A (θx), A (θy) may also be different from each other.

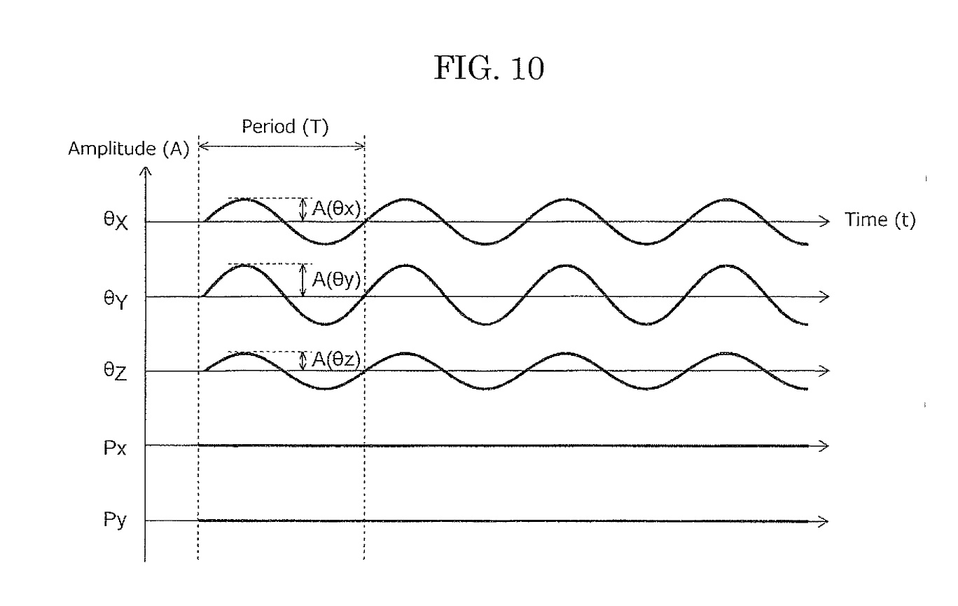

In the example illustrated in FIG. 10, in addition to the motors 20, 22, the motor 24, which is configured to swing the culture bag 100 about the third bag axis Zb, is also used. That is, the rotation angles θx, θy, θz periodically change and the position in the X axis direction Px and the position in the Y axis direction Py are kept zero (the origin).

Similar to the example illustrated in FIG. 9, because the rotation angles θx, θy of the culture bag 100 created by the motors 20, 22 change in the same period and the same phase but in different amplitudes A (θx), A (θy), the culture fluid CF flows within the circularly annular culture space 106 in one circumferential circulation direction R1. However, the rotation angle θz of the culture bag 100 generated by the motor 24 periodically changes about the origin to thereby increase or decrease velocity of the culture fluid CF flowing in one circumferential circulation direction R1. This makes a difference in flow velocity within the culture fluid CF in the circumferential circulation direction R1. This difference in flow velocity causes a turbulent flow within the culture fluid CF. As a result, the culture fluid CF is stirred.

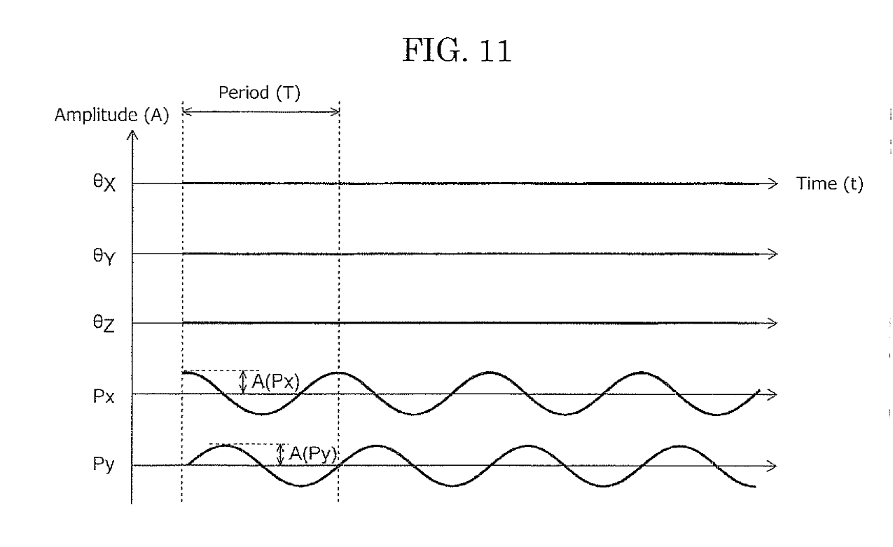

In the example illustrated in FIG. 11, only the actuator 26, which is configured to parallelly move the culture bag 100 in the X axis direction, and the actuator 28, which is configured to parallelly move the culture bag 100 in the Y axis direction, are used. That is, the rotation angles θx, θy, θz are kept zero (the origin), and the position in the X axis direction Px and the position in the Y axis direction Py periodically change. That is, the culture bag 100 reciprocates in the X and Y axes directions.

The position in the X axis direction Px and the position in the Y axis direction Py change in the approximately same period and the approximately same amplitudes A (Px), A (Py). Moreover, the phases are shifted from each other by ¼ of a period. Therefore, the culture bag 100 parallelly moves in an approximate circular orbit. As a result, the culture fluid CF circumferentially circulates within the circularly annular culture space 106 in one circumferential circulation direction R1 at the approximately constant rate.

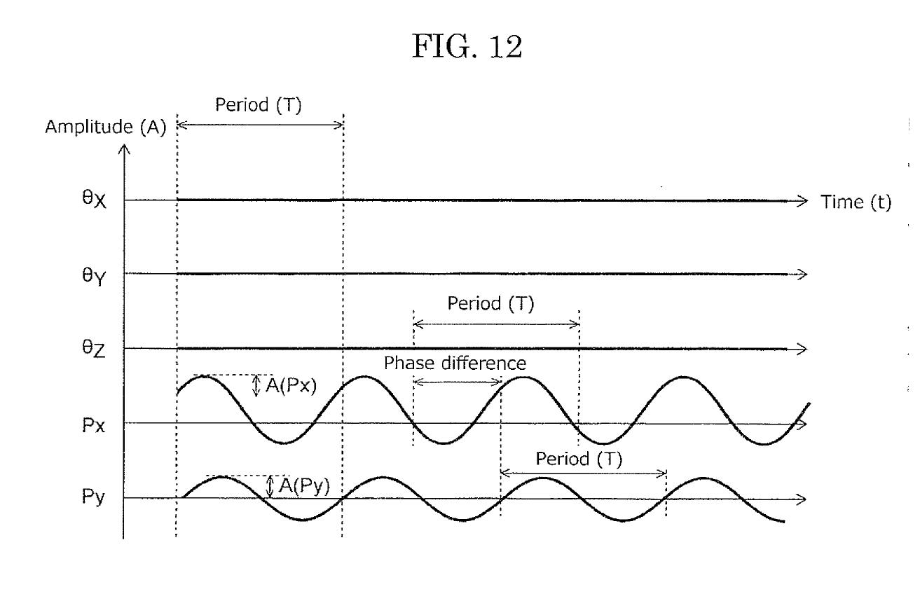

In an example illustrated in FIG. 12, similar to the example illustrated in FIG. 11, only the actuator 26, which is configured to parallelly move the culture bag 100 in the X axis direction, and the actuator 28, which is configured to parallelly move the culture bag 100 in the Y axis direction, are used. That is, the rotation angles θx, θy, θz are kept zero (the origin), and the position in the X axis direction Px and the position in the Y axis direction Py periodically change.

The position in the X axis direction Px and the position in the Y axis direction Py change in the approximately same period, but in different amplitudes A (Px), A (Py) and phases. The position in the X axis direction Px oscillates about a position offset from the origin. Therefore, the culture bag 100 parallelly moves in an elliptical orbit. As a result, the culture fluid CF circumferentially circulates within the circularly annular culture space 106 in one circumferential circulation direction R1. However, the culture bag 100 parallelly moves in an elliptical orbit, so that velocity of the culture fluid CF varies with positions in the circumferential circulation direction R1. This makes a difference in flow velocity within the culture fluid CF in the circumferential circulation direction R1. This difference in flow velocity causes a turbulent flow within the culture fluid CF. As a result, the culture fluid CF is stirred.

Note that, as illustrated in FIGS. 11 and 12, when the actuators 26, 28 are used, the culture bag 100 can parallelly move in a variety of orbits such as an 8-like shape by appropriately modifying change periods, change amplitudes, and phase differences of the position in the X axis direction Px and the position in the Y axis direction Py of the culture bag 100.

Control on the motors 20, 22, 24 and the actuators 26, 28 so as to circumferentially circulate the culture fluid CF within the circularly annular culture space 106 of the culture bag 100 may be changed over time, that is, in accordance with the degree of progress in culturing.

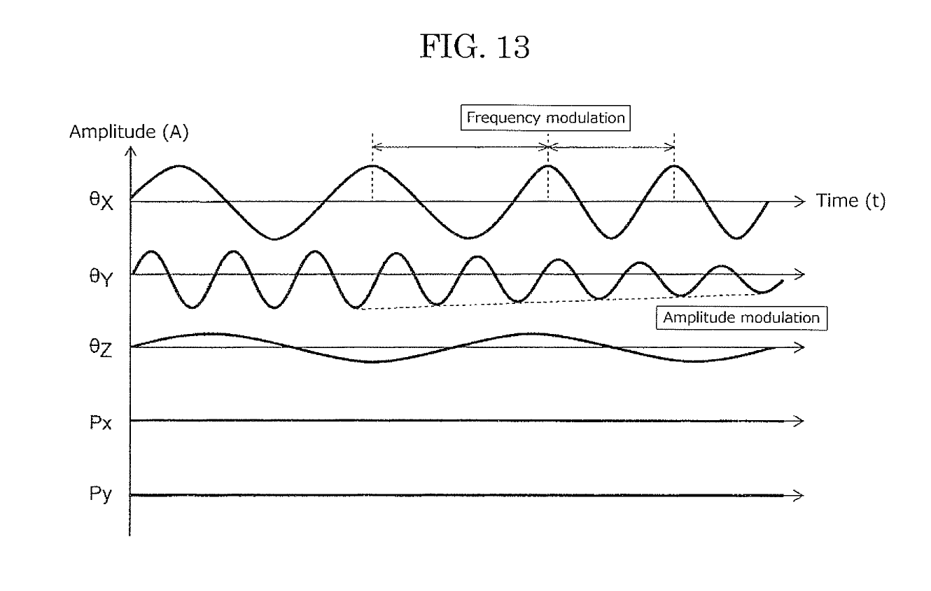

In an example illustrated in FIG. 13, the rotation angle θx about the first bag axis Xb of the culture bag 100 is modulated in change frequency. Specifically, the frequency increases over time. The rotation angle θy about the second bag axis Yb is also modulated in change amplitude. Specifically, the amplitude decreases over time. Note that, the modulations in frequency and amplitude may be a step modulation in which the frequency and the amplitude are changed at each predetermined timing over the entire culture period or a sweep modulation in which the frequency and the amplitude are continuously changed until the end of a culture period or reaching the predetermined timing.

Thus, culturing can be facilitated in some types of culturing by changing control on the motors 20, 22, 24 and the actuators 26, 28 over time. For example, cell proliferation can be facilitated.

Thus, the culture fluid CF within the circularly annular culture space 106 of the culture bag 100 can circumferentially circulate in various modes by selectively using the motors 20, 22, 24 and the actuators 26, 28. Therefore, the mode in which the culture fluid CF circumferentially circulates can be appropriately selected depending on the type of culturing.

According to the present invention described above, waves of a culture fluid CF, the waves creating bubbles and shear stress which may damage a culture target, can be suppressed from being created without decreasing culture efficiency in a culturing which is performed while the culture fluid CF is flowing in a culture bag 100.

Specifically, as illustrated in FIG. 3, because the culture space 106 in which the culture fluid CF is contained and cultured is an endless circumferential circulation space, in particular, a circularly annular space, so that the culture CF can circumferentially circulate.

Collision between an inner wall surface of the culture space 106 and waves of the culture fluid CF can be suppressed by allowing the culture fluid CF to circumferentially circulate (regulating a flow direction to the circumferential circulation direction R1) than the case in which a flow direction changes in an unregulated manner. Specifically, collision due to a rapid change of the flow direction of the culture fluid CF (e.g., reversal of the flow direction) can be suppressed from occurring. This suppresses bubbles and shear stress from being creating to an extent that the culture target (e.g., cells) is not damaged.

Smaller wave of the culture fluid CF are created by allowing the culture fluid CF to circumferentially circulate (regulating a flow direction to the circumferential circulation direction R1) than the case in which a flow direction changes in an unregulated manner. That is, waves of the culture fluid, which are big enough to create bubbles and shear stress which may damage the culture target (e.g., cells), are suppressed from occurring.

Moreover, because the culture space 106 in which the culture fluid CF flows is an endless circumferential circulation space in which the culture fluid CF can circumferentially circulate, a region of which flow velocity is almost zero (so-called stagnancy) is suppressed from occurring in the culture fluid CF. Therefore, the culture target is suppressed from aggregating in the region of which flow velocity is almost zero. As a result, the culture target is suppressed from being damaged.

Note that, as supplementary information, the culture fluid CF flows along the inner wall surface of the culture space 106 by allowing the culture fluid CF to circumferentially circulate (regulating a flow direction to the circumferential circulation direction R1). This makes a difference in flow velocity in the proximity of the inner wall surface of the culture space 106 due to viscosity of the culture fluid CF. The difference in flow velocity causes flow separation to thereby create a lot of small eddies (microeddies). These microeddies are repeatedly created and eliminated and contribute to stirring of the culture fluid CF. Therefore, according to the present embodiment, in order to suppress the bubbles and the shear stress which may damage the culture target (that is, big waves of the culture fluid) from occurring, the culture fluid CF is allowed to circumferentially circulate to thereby keep a liquid surface of the culture fluid CF smooth. Meanwhile, microeddies are created for stirring in the culture fluid CF.

The present invention has been described with reference to the above-mentioned embodiments, but embodiments of the present invention are not limited thereto.

For example, in the case of the above-mentioned embodiments, the culture space 106 of the culture portion 102 of the culture bag 100 is circularly annular, but not limited thereto.

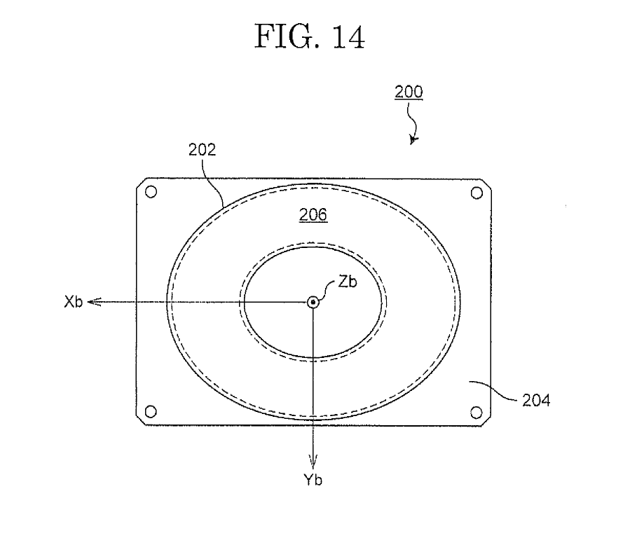

For example, in the case of another embodiment, as illustrated in FIG. 14, a culture bag 200 includes a culture portion. 202 having elliptically annular culture space 206.

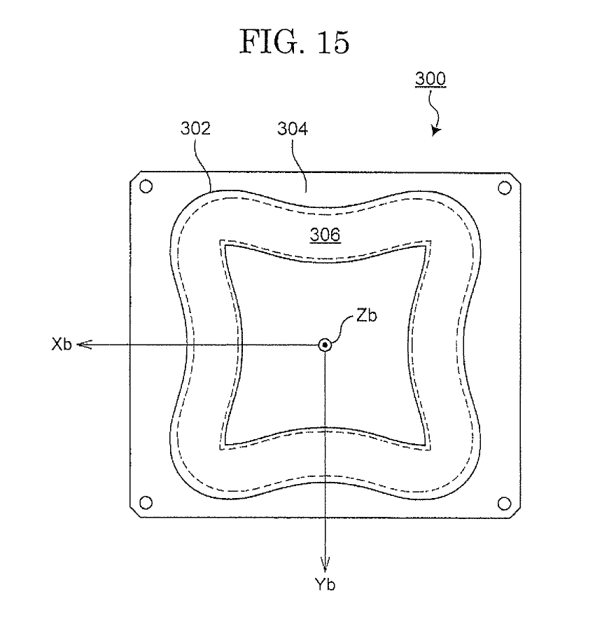

For example, in the case of additional another embodiment, as illustrated in FIG. 15, a culture bag 300 includes a culture portion 302 having an approximately quadrilaterally annular culture space 306. Specifically, the culture space 306 has a quadrilateral shape in which each of four sides is convexly curved toward the center.

In the case of the culture bag 200 illustrated in FIG. 14 and the culture bag 300 illustrated in FIG. 15, the culture fluid has relatively high velocity at portions of the culture spaces 206, 306 which have a relatively large curvature, but relatively low velocity at portions of the culture spaces 206, 306 which have a relatively small curvature. This makes a difference in flow velocity within the culture fluid in the circumferential circulation direction of the culture space. This difference in flow velocity causes a turbulent flow, so that the culture fluid is stirred to a greater extent compared to in the circularly annular culture space.

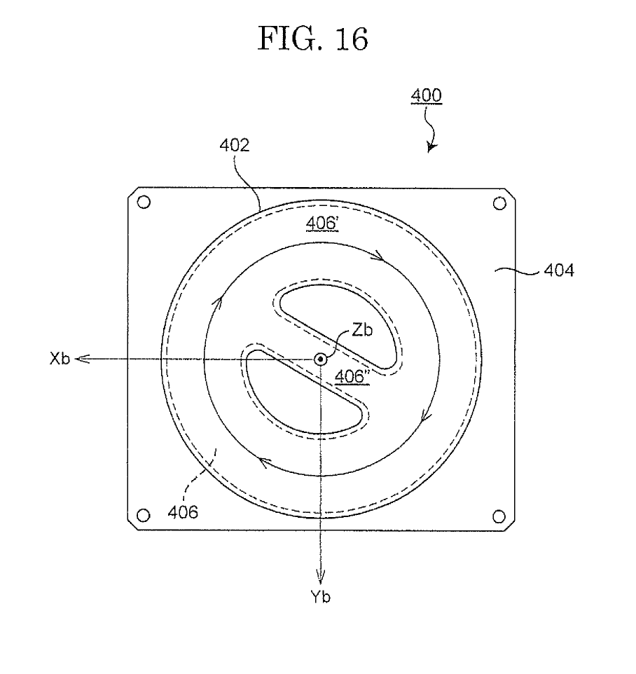

Moreover, for example, in the case of a different embodiment, as illustrated in FIG. 16, a culture bag 400 includes a culture portion 402 having a culture space 406 which includes a circularly annular space portion 406′ and a linear space portion 406″ which extends in a radial direction of the circularly annular space portion 406′ and of which both ends are coupled to the space portion 406′.

Broadly speaking, the culture space according to embodiments of the present invention only has to include an endless circumferential circulation space in which the culture fluid contained therein can circumferentially circulate as a whole or in part. Therefore, the culture space may be a three-dimensional shape crossing each other in a three-dimensional manner such as an “8”-like shape. However, taking formation and maintenance of a circumferentially circulating flow of the culture fluid as well as productivity of the culture bag into consideration, the culture space has preferably an annular shape, particularly preferably a circularly annular shape.

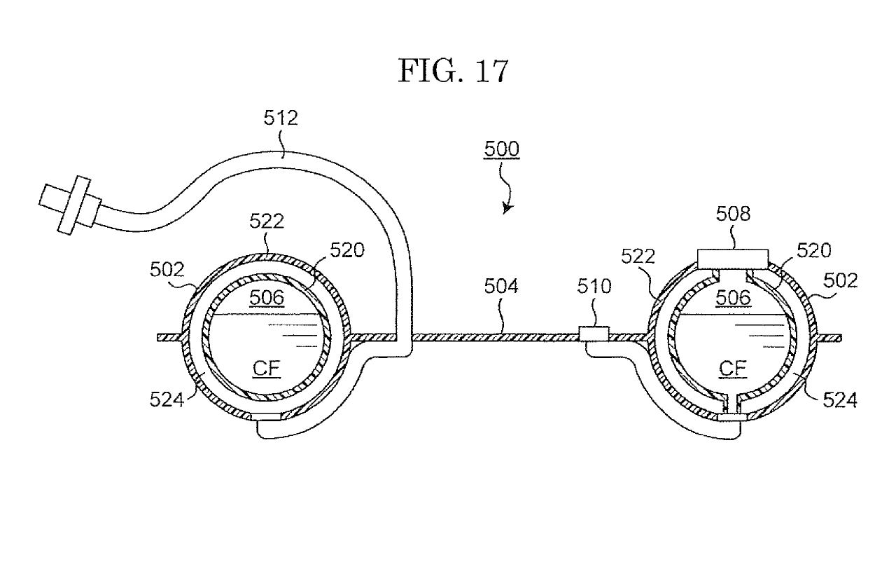

The culture portion of the culture bag may have a double bag structure. For example, a culture portion 502 of a culture bag 500 according to a further different embodiment illustrated in FIG. 17 includes a circularly annular inner bag portion 520 having a circular longitudinal section and a circularly annular outer bag portion 522 containing the inner bag portion 520 and having a circular longitudinal section. An inner space of the inner bag portion 520 is the culture space 506 containing the culture fluid CF.

Oxygen and carbon dioxide are supplied via a gas supplying port 512 into a space (gas-containing space) 524 between the inner bag portion 520 and the outer bag portion 522.

The oxygen and the carbon dioxide which have been supplied into the gas-containing space 524 between the inner bag portion 520 and the outer bag portion 522 pass through the inner bag portion 520 into the culture space 506 within the inner bag portion 520. For this purpose, the inner bag portion 520 is configured to contain the culture fluid in the culture space 506 and to pass a gas from the gas-containing space 524 into the culture space 506. For example, the inner bag portion 520 has a plurality of holes each having an aperture area so as to be gas-permeable but not to be permeable to the culture fluid. For example, the inner bag portion 520 may be made of a gas-permeable film.

Because the oxygen and the carbon dioxide have passed through the inner bag portion 520, the oxygen and the carbon dioxide are supplied into the culture fluid CF within the culture space 506 in a form of microbubbles. As a result, the oxygen and the carbon dioxide are easily dissolved into the culture fluid CF. This reduces the necessity to make the culture fluid flow (the culture fluid only has to flow to an extent necessary for facilitating culturing), making it more difficult to create waves of the culture fluid CF, the waves creating bubbles and shear stress which may damage the culture target.

Moreover, in the case of the above-mentioned embodiment, as illustrated in FIGS. 4 and 5, the culture space 106 of the culture bag 100 has a circular longitudinal section, but embodiments of the present invention are not limited thereto.

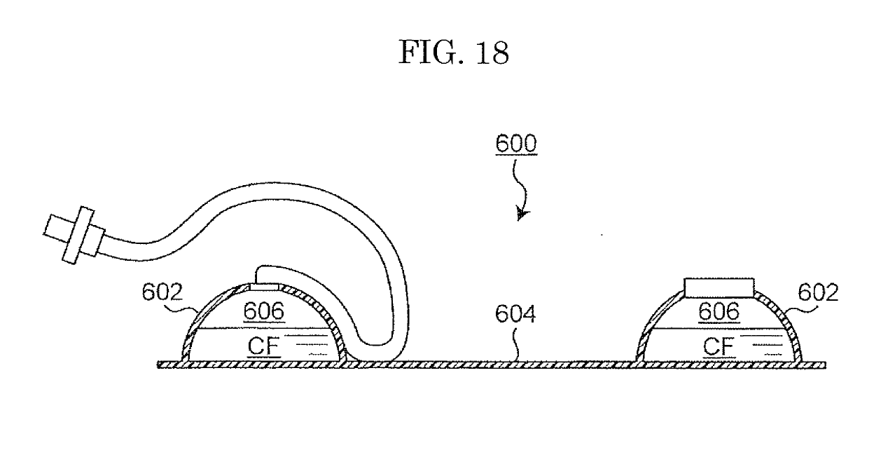

For example, a culture bag 600 according to a further different embodiment illustrated in FIG. 18 includes a culture portion 602 having a culture space 606 which has a semi-circular longitudinal section. Thus, the culture space can have a variety of longitudinal section shapes. For example, the longitudinal section may be elliptical, semi-circular, or polygonal. However, in order for the culture fluid to smoothly flow in a longitudinal section circumferential direction and along an inner surface of the culture space, that is, in order to suppress creation of shear stress due to a rapid change of a flow direction, the inner surface is preferably a continuously curved surface.

In the case of the above-mentioned embodiments, as illustrated in FIGS. 2 to 5, the culture bag 100 includes the circularly annular culture space 106 in advance. However, embodiments of the present invention are not limited thereto.

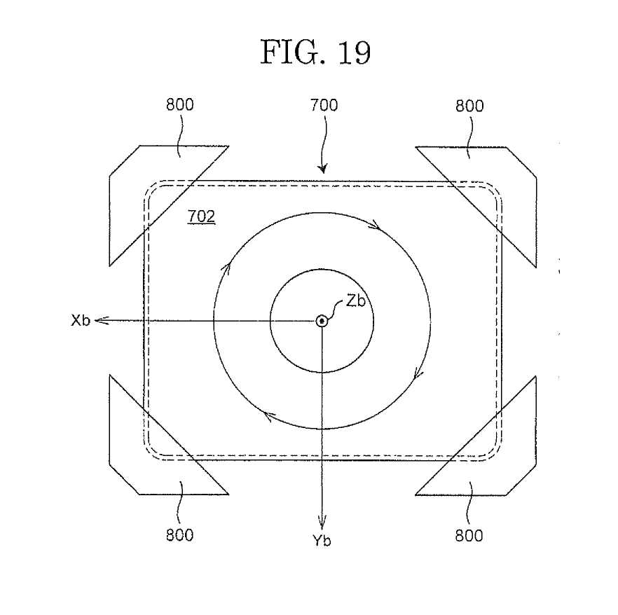

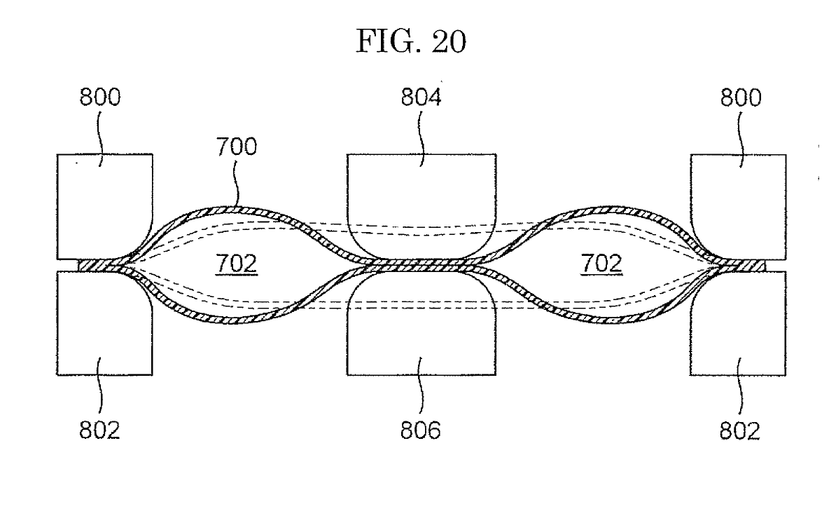

For example, FIGS. 19 and 20 schematically illustrate a configuration of a culture device according to another embodiment.

The culture device illustrated in FIGS. 19 and 20 is configured to deform a rectangular culture space 702 of a rectangular culture bag 700 into an annular space. Specifically, the culture device includes a pair of clamp bars 800, 802, which are configured to clamp each of corners of the rectangular culture bag 700, and a pair of clamp bars 804, 806, which are configured to clamp the center of the culture bag 700.

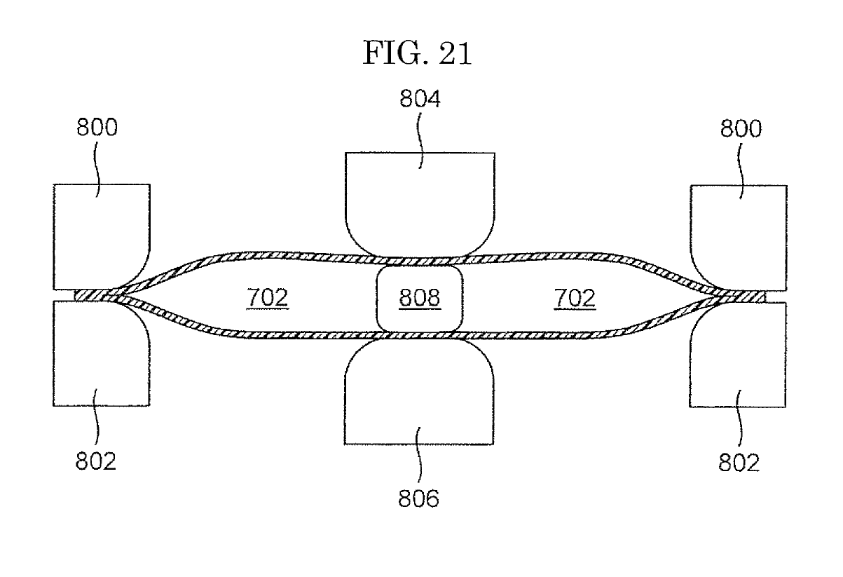

The culture space 702 is deformed into an annular space by pressing a central portion of the culture bag 700 by means of each of the pair of clamp bars 804, 806 to thereby bring opposed inner surfaces of the central portion of the culture space 702 into contact with each other. That is, the pair of clamp bars 804 serves as a culture space deforming portion configured to deform the culture space into an annular space. While keeping the thus-deformed state, the culture device changes a position and a posture of the culture bag 700 so that the culture fluid circumferentially circulates in the annular culture space 702. In this case, the culture bag is more easily produced than a culture bag having an annular culture space in advance. Note that, as illustrated in FIG. 21, a cylindrical block 808 may be inserted into the center of the culture space 702 of the culture bag 700 and be clamped by the pair of clamp bars 804, 806 (the block 808 also serves as the culture space deforming portion).

Note that, the culture bag 100 according to the above-mentioned embodiment can be used not only in the culture device 10 illustrated in FIG. 1 but also in commonly used devices. That is, the culture bag 100 can be used in any device, as long as the device can change a position and a posture of the culture bag 100 so that the culture fluid CF circumferentially circulates within the endless culture space 106 of the culture bag 100.

Finally, as supplementary information, in embodiments according to the present invention, the culture fluid can circumferentially circulate by flowing within an endless circumferential circulation space in which the culture fluid can circumferentially circulate (e.g., a doughnut-like space as illustrated in FIGS. 2 to 5). However, the culture fluid can also circumferentially circulate in other spaces than the endless space, e.g., in a disk-like space. In this case, at the center of the circumferential circulation, there would be a flow of the culture fluid of which flow velocity is almost zero. Therefore, as described above, the culture target aggregates at the center of the circumferential circulation at which the flow velocity is almost zero, to thereby damage the culture target. As a result, culture efficiency is decreased.

Therefore, in the present application, the phrase “endless circumferential circulation space in which a culture fluid can circumferentially circulate” denotes a space in which the culture fluid can circumferentially circulate and which includes an inner surface regulating movement of the culture fluid toward the center of the circumferential circulation (e.g., a center-side inner circumferential surface of the circularly annular culture space 106 illustrated in FIG. 3 or an outer circumferential surface of the cylindrical block 808 illustrated in FIG. 21).

As described above, the embodiments have been described as exemplifications of the technique in the present invention. To this end, the accompanying drawings and detailed description have been provided. Accordingly, the components described in the accompanying drawings and detailed description can include not only components essential to solve the problem but also components unessential to solve the problem, for the purpose of merely exemplifying the above technique. Hence, those unessential components should not directly be construed as being essential from the fact that those unessential components are described in the accompanying drawings and detailed description.

Since the above embodiments are for exemplifying the technique in this invention, various modifications, replacements, additions, omissions can be made without departing from the scope of claims and equivalents thereof.

The disclosed contents of the specification, the drawings, and the claims of Japanese Patent Application No. 2015-232251 filed on Nov. 27, 2015 are incorporated herein by reference as their entirety.

1. A culture bag comprising
a culture portion which has a culture space configured to contain and culture a culture fluid;
wherein the culture space is an endless circumferential circulation space in which the culture fluid can circumferentially circulate.

2. The culture bag according to claim 1, wherein the culture space is annular.

3. The culture bag according to claim 2, wherein a longitudinal section perpendicular to a circumferential circulation direction of the culture space is circular.

4. The culture bag according to claim 1, wherein the culture portion has a double bag structure and includes an inner bag portion and an outer bag portion configured to contain the inner bag portion;
wherein an inner space of the inner bag portion is the culture space;
wherein a space between the inner bag portion and the outer bag portion is a gas-containing space configured to contain a gas; and
wherein the inner bag portion is configured to contain the culture fluid in the inner space and be gas-permeable.

5. A culture device comprising:
a culture bag which comprises a culture portion having a culture space configured to contain and culture a culture fluid, the culture space being an endless circumferential circulation space in which the culture fluid can circumferentially circulate;
a stage configured to hold the culture bag; and
a stage driving portion configured to change a position and a posture of the stage so that the culture fluid circumferentially circulates in the culture space of the culture bag.

6. A culture device comprising:
a culture bag having a culture space configured to contain and culture a culture fluid;
a stage configured to hold the culture bag;
a culture space deforming portion configured to deform the culture space into an annular culture space; and
a stage driving portion configured to change a position and a posture of the stage so that the culture fluid circumferentially circulates in the annular culture space of the culture bag after deformation.
